(19) United States
(12) Patent Application Publication
(10) Pub. No.: US 2005/0008664 A1
(43) Pub. Date: Jan. 13, 2005

(54) Compositions and methods related to lipid:emodin formulations

(75) Inventors: David Claxton, Hummelstown, PA (US); Robert A. Newman, Houston, TX (US); Gabriel Lopez-Berestein, Bellaire, TX (US)

Correspondence Address: FULBRIGHT & JAWORSKI L.L.P., 600 CONGRESS AVE., SUITE 2400, AUSTIN, TX, 78701, US

(73) Assignees: Board of Regents, The University of Texas System; The Penn State Research Foundation

(21) Appl. No.: 10/730,361

(22) Filed: Dec. 8, 2003

Related U.S. Application Data
(60) Provisional application No. 60/431,422, filed on Dec. 6, 2002.

(51) Int. Cl. A61K 31/12; A61K 7/00

(52) U.S. Cl. 424/401; 514/680

(57) ABSTRACT

The present invention concerns the use of methods and compositions to provide an improved lipid:emodin formulation for the treatment of leukemias expressing bcr-abl and other cancer with elevated tyrosine kinase activity.

### BACKGROUND OF THE INVENTION

This application claims priority to U.S. Provisional Patent Application Ser. No. 60/431,422 filed on Dec. 6, 2002, which is incorporated herein in its entirety by reference.

1. Field of the Invention

The present invention relates generally to the fields cancer therapeutics. More particularly, it concerns lipid formulations of emodin.

2. Description of Related Art

Emodin (3-methyl-1, 6, 8, trihydroxyanthran-quinone) is a naturally occurring substance that has been used in traditional Chinese medicine for some time. Its' mechanism of action is as a tyrosine kinase inhibitor, for example restricting the activity of p56^{lck} protein tyrosine kinase. It has also been shown to inhibit the growth of cancer cells, including the growth of lymphocytic leukemia and HL-60 cells. In addition, emodin has been shown to inhibit Her2-Neu tyrosine kinase activity and has demonstrated in vivo activity against Her2-Neu transformed NIH3T3 cells. Emodin also inhibits the tyrosine kinase activity of bcr-abl, which is critical in leukemogenesis and drug resistance. Patients who have leukemias expressing bcr-abl typically receive a poor prognosis. There is a need for a therapeutic agent for the treatment of patients with bcr-able leukemias and other cancer with elevated tyrosine kinase activity.

However, current emodin formulations are alkaline preparations and present the problem of a high pH solution being injected into animals. Lowering the pH of a formulation beyond the solubility of emodin forms a precipitate in the solution. Because of this limited solubility at approximately neutral pH the present emodin formulations are not optimal for use in animals or humans.

### SUMMARY OF THE INVENTION

One potential method to reduce the problems associated with emodin administration would be the use of a drug delivery system. The lipid based format is a useful one for drug delivery in vivo. This, in essence, involves attaining a high concentration and/or long duration of drug action at a target (e.g. a tumor) site where beneficial effects may occur, while maintaining a low concentration and/or reduced duration at other sites where adverse side effects may occur (Juliano, et al., 1980). In certain aspects the lipid associated emodin may be diffuse from the lipid in a time dependent manner. Lipid-association of drug may be expected to impact upon the problems of controlled drug delivery since lipid association radically alters the pharmacokinetics, distribution and metabolism of drugs.

In various embodiments, compositions include emodin, or a derivative thereof, associated with a lipid. Emodin derivatives or emodin-like molecules are those compounds that exhibit similar characteristics to those of emodin with regard to tyrosine kinase inhibition and the inhibition of cell transformation. The emodin or emodin derivative may be, for example, emodin, emodin-8-O-D-glucoside, chrysophanic acid, gluco-chrysophanic acid, physcion, or physcion-8-O-D-glucoside. The lipid may include a variety of lipids known in the art, in particular dimyristol phosphatidyl choline or dimyristol phosphatidyl glycerol. The weight/weight ratio of lipid to emodin may be approximately 5:1 to approximately 30:1. In certain embodiments the ratio of lipid to emodin is approximately 5:2. The weight/weight ratio may include 5, 6, 7, 8, 9, 10, 11, 12, 13, 14, 15, 16, 17, 18, 19, 20, 21, 22, 23, 24, 25, 26, 27, 28, 29, or 30 lipid to 0.1, 0.2, 0.3, 0.4, 0.5, 0.6, 0.7, 0.8, 0.9, 1, 2, 3, 4, 5, 6, 7, 8, 9, or 10 emodin or emodin derivative.

In some embodiments the composition includes a solubilizing agent. The solubilizing agent may be a non-ionic detergent in an amount of approximately 0.05, 0.06.0.07, 0.08, 0.09, 0.1, 0.15, 0.2, 0.3, 0.4, 0.5, 0.6, 0.7, 0.8, 0.9, 1, 2, 3, 4, 5, 6, 7, 8, 9, 10, 11, 12, 13, 14, or 15 percent weight/weight of the formulation prior to lyophilization. In certain embodiments the amount of non-ionic detergent is approximately 0.08 percent weight/weight of the formulation prior to lyophilization. The non-ionic detergent may be tween, tween 20 or similar detergents. In other embodiments, the solubilizing agent is soybean or peanut oils. In some embodiments the solubilizing agent is a β-hydroxylated compound.

Various embodiments of the invention include methods of preparing a composition, comprising the step of admixing emodin or a derivative thereof with a lipid in a solvent. The method may also include admixing a solubilization agent. In certain embodiments the lipid is dimyristolphosphatidylcholine (DMPC). The ratio of lipid to emodin and the percentage of solubilization agent is similar to that described above, which is incorporated here by reference. In various embodiments the solvent is tertiary butanol. The solubilization agent includes soy or peanut oils and β-hydroxylated compound. In certain embodiments the solubilization agent is tween 20.

The method may further comprise lyophilizing the composition. In various embodiments methods may further comprise reconstituting the lyophilized composition in a solvent. The solvent may be an aqueous solvent, for example a saline solution. In certain embodiments the saline solution is a 0.9% saline solution.

Various embodiments include methods for treating cancer in a subject comprising administering to said subject a formulation comprising emodin associated with a lipid, wherein a tyrosine kinase activity in cancer cells is inhibit in said subject. In some embodiments the cancer is a hematopoetic cancer, for example leukemia or lymphoma. Emodin may be provided in a dose of approximately 1 mg/Kg of body weight to approximately 500 mg/Kg of body weight. The methods of the invention may include administration of the lipid:emodin formulation by injection, for example intravascular injection. It is also contemplated that a lipid:emodin formulation may be administered more than 1, 2, 3, 4, 5, 6, 7, 8, 9, 10 or more times over a period of hours, days weeks, months and even years.

It is contemplated that any method or composition described herein can be implemented with respect to any other method or composition described herein.

The use of the word “a” or “an” when used in conjunction with the term “comprising” in the claims and/or the specification may mean “one,” but it is also consistent with the meaning of “one or more,” “at least one,” and “one or more than one.”

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 shows an example of lipid:emodin treatment of a leukemic mouse model. Leukemogenic 32D-bcr-abl cells were transplanted on day 0 into a congenic strain of mice CEH/HEJ. An exemplary lipid:emodin formulation was administered to the mice to study its effectiveness as a treatment for leukemia. Animals receiving no treatment died between 18 and 22 days. Treated animals received 1 or 2 injections of lipid:emodin at 8 mg/Kg body weight beginning on day 1. Lipid:emodin treated animals show a significant survival fraction, which is larger in the group receiving 2 injections as compared to the group receiving 1 injection.

FIG. 2 shows an exemplary study of DMSO:emodin in Tet regulatable P210 cells, a three day assay.

FIG. 3 shows an exemplary study of the effects of emodin on tyrosine phosphorylation of bcr-abl.

FIG. 4 shows an exemplary study of the effects of emodin on tyrosine phosphorylation of bcr-abl in K562 cells.

FIG. 5 shows an exemplary animal study of DMSO:emodin administration in C3H mice.

FIG. 6 shows the diffusion of emodin from liposomes (ELP) over time. The initial loading concentration of emodin in the liposomes was 2.8 mg/mL.

### DESCRIPTION OF ILLUSTRATIVE EMBODIMENTS

Certain aspects of the invention include an improved formulation of the drug 3-methyl-1, 6, 8 trihydroxyanthra-quinone (emodin) or derivatives thereof. In particular embodiments, emodin is provided in a lipid formulation. Typically, a lipid formulation improves the solubility of emodin in a pharmaceutical formulation. The lipid formulation may also include a solubilizing agent. The soubilizing agent may include, but is not limited to β-hydroxlated compound(s), e.g., soy or peanut oils, as well as a non-ionic detergent(s), e.g., tween 20. Suitable therapeutic lipid:emodin formulations in accordance with the present invention include emodin and/or derivatives thereof.

Lipid formulations may include lipid carrier particles, such as liposomes, that can be formed by methods that are well known in this field. Suitable phospholipid compounds include, but are not limited to phosphatidyl choline, phosphatidic acid, phosphatidyl serine, sphingolipids, sphingomyelin, cardiolipin, glycolipids, gangliosides, cerebrosides, phosphatides, sterols, and the like. More particularly, the phospholipids which can be used include, but are not limited to dimyristoyl phosphatidyl choline, egg phosphatidyl choline, dilauryloyl phosphatidyl choline, dipalmitoyl phosphatidyl choline, distearoyl phosphatidyl choline, 1-myristoyl-2-palmitoyl phosphatidyl choline, 1-palmitoyl-2-myristoyl phosphatidyl choline, 1 -palmitoyl-2-stearoyl phosphatidyl choline, 1-stearoyl-2-palmitoyl phosphatidyl choline, dioleoyl phosphatidyl choline, dimyristoyl phosphatidic acid, dipalmitoyl phosphatidic acid, dimyristoyl phosphatidyl ethanolamine, dipalmitoyl phosphatidyl ethanolamine, dimyristoyl phosphatidyl serine, dipalmitoyl phosphatidyl serine, brain phosphatidyl serine, brain sphingomyelin, dipalmitoyl sphingomyelin, and distearoyl sphingomyelin.

In addition, other lipids, steroids, cholesterol, and the like may be intermixed with the phospholipid components to confer certain desired and known properties on the resultant liposomes. Further, synthetic phospholipids containing either altered aliphatic portions, such as hydroxyl groups, branched carbon chains, cyclo derivatives, aromatic derivatives, ethers, amides, polyunsaturated derivatives, halogenated derivatives, or altered hydrophilic portions containing carbohydrate, glycol, phosphate, phosphonate, quaternary amine, sulfate, sulfonate, carboxy, amine, sulfhydryl, imidazole groups and combinations of such groups, can be either substituted or intermixed with the phospholipids, and others known to those skilled in the art.

A suitable solubilization agent may be included in the lipid:emodin formulation. Non-ionic detergents or β-hydroxylated products/compounds are preferred solubilization agents, with tween 20 and soybean oil as specific examples. Other suitable solubilization agents include peanut oils, sterols, such as cholesterol, fatty alcohols, fatty acids, fatty acids esterified to a number of moieties, such as polysorbate, propylene glycol, mono- and diglycerides, and polymers such as polyvinyl alcohols.

Prior to lyophilization, emodin, lipid(s), and/or solubilization agent can be dissolved in an organic solvent, such as tertiary butanol (t-butanol) or the like. Lyophilization to form a preliposomal powder can be performed using commercial apparatus which is known to persons skilled in this field. After lyophilization, the powder can be reconstituted, e.g., as liposomes or lipid formulations, by adding a pharmaceutically acceptable carrier, such as sterile water, saline solution (e.g., a 0.9% saline solution), or dextrose solution, with agitation, and optionally with the application of heat.

An exemplary formulation, which can be dissolved in t-butanol, is a lipid:emodin ratio of 5:2 with a final concentration of 0.08% w/w of tween 20.

A composition of the present invention is preferably administered to a patient parenterally, for example by intravenous, intraarterial, intramuscular, intralymphatic, intraperitoneal, subcutaneous, intrapleural, or intrathecal injection. Administration could also be by topical application or oral dosage. Preferred dosages are between 40 to 200 mg/m². The dosage may range from 20, 25, 30, 25, 40, 45, 50, 55, 60, 65, to 70, 75, 80, 85, 90, 100, 125, 150, 200, 250, 300 mg/m² include all ranges therebetween. The dosage is preferably repeated on a timed schedule until tumor or disease regression, stasis or disappearance has been achieved, and may be used in conjunction with other forms of cancer therapy such as surgery, radiation, or chemotherapy with other agents.

The present invention is useful in the treatment of cancer, in particular cancer associated with elevated tyrosine kinase activity, including for example: hematological malignancies such as leukemia and lymphoma, carcinomas such as breast, lung, and colon.

### I. Structural Properties of Emodin and Anthraquinone-based Emodin-like Compounds

Emodin (3-methyl-1, 6, 8 trihydroxyanthra-quinone) belongs to a group of compounds that are structurally based upon the structure of anthraquinone shown in Table 1, to which various R groups may be added. A wide variety of anthraquinones exist in nature (Yeh et al., 1988; Kupchan and Karim, 1976; Jayasuriya et al., 1992). Structure B in Table 1 is emodin itself; C is emodin-8-O-D-glucoside; D is chrysophanic acid; E is gluco-chrysophanic acid; F is physcion; and G is physcion-8-O-D-glucoside. The emodin-like compounds of structures A, C and D-G are only exemplary forms of emodin-like compounds (emodin derivatives) that may be used in the present invention. Numerous other emodin analogues are available as shown in Table 1 and as described by, e.g., Yeh et al., 1988; Kupchan and Karim, 1976; Jayasuriya et al., 1992.

The first group (group A: Table 1) is comprised of compounds which are structurally related to emodin, only replace CH₃ group with different other group at C₃ of emodin, their inhibitory activities of tyrosine phosphorylation of p185^{neu} are in following order CH₃>C═NOCH₃>CHNOH>CH₂OH>CONH₂>COOH and inhibitory activities for proliferation of cells are CH₃>CHNOH>CONH₂>C═NOCH₃>CH₂OH>CH₂OH>COOH, these results indicate that CH₃ group at C₃ position of emodin is, typically, important to retain inhibitory activities of emodin on tyrosine phosphorylation and proliferation.

The second group (Group B: Table 1) also structurally related to emodin, only replace OH group with either H or OCH₃ group at C₆ position of emodin. However, compare with emodin, their inhibitory activity for both tyrosine phosphorylation of p185^{neu} and proliferation of cells are 5-fold lower than emodin.

The third (Group C), after removal OH groups at C₁, C₆ and C₈, and CH₃ group at C₃ of emodin and addition of NH₂ group at C₁ and C₂ of emodin, shows a decrease in activity of emodin. The fifth group (group E) removes the ketone group from C₁₀, and also reduces the activity of emodin.

The fourth group (group D) is structurally similar to the third group, with the exception of a replaced C₉ ketone with either p-acetylamidebenzomethyl group (DK-V-47) or p-aminobenzomethyl group (DK-V-48), DK-V-47 has higher activity than emodin in regard to inhibition of tyrosine phosphorylation of p185^{neu} and proliferation of cancer cells. However, replace the COCH₃ of DK-V-47 with an H group (DK-V-48), DK-V-48 results in a decrease in the activity of DK-V-47. These results suggest that COCH₃ group of DK-V-47 is involved in the maintenance of the activity of DK-V-47.

TABLE 1
Structures of Emodin-like Compounds

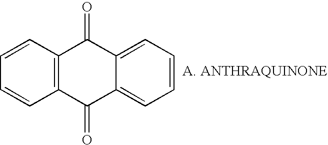

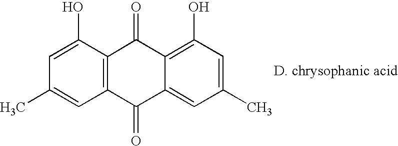

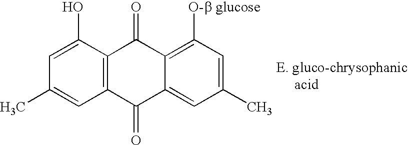

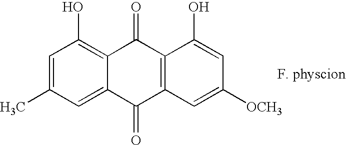

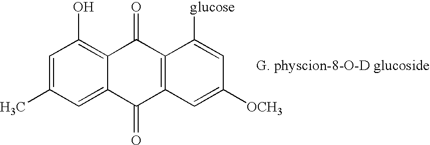

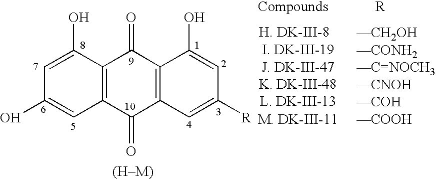

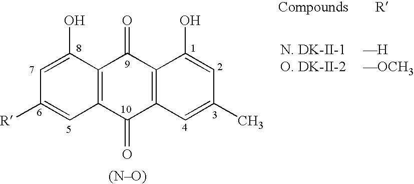

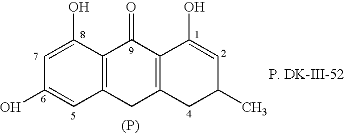

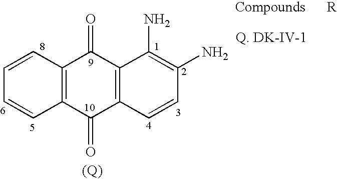

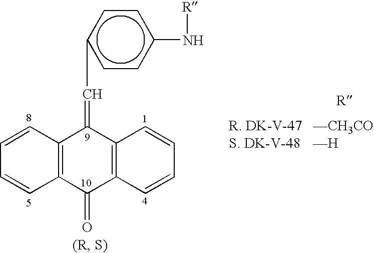

### II. Functional Properties of Emodin and Emodin-like Compounds

Emodin, which was first isolated from polygonum cuspidatum, has been shown to be an inhibitor of a variety of protein tyrosine kinases, for example the protein tyrosine kinase p56^{lck} (Jayasuriya et al.; 1992). Emodin has been reported to be a tyrosine kinase inhibitor that restricts the activity of p56^{lck} kinase by preventing the binding of ATP in vitro (Jayasuriya et al., 1992). Emodin also can inhibit the growth of cancer cells, including lymphocytic leukemia (Kupchan et al., 1976), HL-60 human leukemia cells (Yeh et al., 1988), and ras-transfonned human bronchial epithelial cells (Chan et al., 1993), by an unknown mechanism.

Emodin and emodin-like compounds assist in overcoming the chemoresistance of neu-overexpressing cancer cells by sensitizing these cells to chemotherapeutic agents. The effects of emodin on the tyrosine phosphorylation (e.g. phosphorylation of the neu protein), cellular proliferation, and cellular morphology in cancer cells, as well as the effects of emodin in combination with chemotherapeutic agents has been examined. Emodin has been found to suppressed tyrosine phosphorylation of the neu protein, preferentially inhibited proliferation of neu-expressing lung cancer cells to a surprising level, and sensitized these cells to chemotherapeutic drugs. This suppression of tyrosine phosphorylation is a functional characteristic of emodin-like compounds.

Emodin and emodin-like compounds have been shown to suppress the tyrosine kinase activity of neu-overexpressing human breast cancer cells, suppresses their transforming ability, and induces their differentiation. Further, emodin also suppresses tyrosine phosphorylation of neu protein in lung cancer cells and preferentially inhibits growth of these cells. Emodin is also able to sensitize lung cancer cells that overexpress neu to the chemotherapeutic agents cisplatin, doxorubicin, and VP16. This suggests that the tyrosine kinase activity of p185^{neu} is required for the chemoresistant phenotype of neu overexpressing cancer cells.

### III. Therapeutically Effective Amounts of Emodin and Emodin-like Compounds

A therapeutically effective amount of an emodin and/or emodin-like tyrosine kinase inhibitor that is formulated with a lipid carrier varies depending upon the host treated and the particular mode of administration. In one embodiment of the invention the dose range of an emodin-like tyrosine kinase inhibitor used will be about 0.5 mg/kg body weight to about 500 mg/kg body weight. The term “body weight” is applicable when an animal is being treated. When isolated cells are being treated, “body weight” as used herein should read to mean “total cell weight”. The term “total weight” may be used to apply to both isolated cell and animal treatment. All concentrations and treatment levels are expressed as “body weight” or simply “kg” in this application are also considered to cover the analogous “total cell weight” and “total weight” concentrations. However, those of skill will recognize the utility of a variety of dosage range, for example, 1 mg/kg body weight to 450 mg/kg body weight, 2 mg/kg body weight to 400 mg/kg body weight, 3 mg/kg body weight to 350 mg/kg body weight, 4 mg/kg body weight to 300 mg/kg body weight, 5 mg/kg body weight to 250 mg/kg body weight, 6 mg/kg body weight to 200 mg/kg body weight, 7 mg/kg body weight to 150 mg/kg body weight, 8 mg/kg body weight to 100 mg/kg body weight, or 9 mg/kg body weight to 50 mg/kg body weight. Further, those of skill will recognize that a variety of different dosage levels will be of use, for example, 1 mg/kg, 2 mg/kg, 3 mg/kg, 4 mg/kg, 5 mg/kg, 7.5 mg/kg, 10, mg/kg, 12.5 mg/kg, 15 mg/kg, 17.5 mg/kg, 20 mg/kg, 25 mg/kg, 30 mg/kg, 35 mg/kg, 40 mg/kg, 45 mg/kg, 50 mg/kg, 60 mg/kg, 70 mg/kg, 80 mg/kg, 90 mg/kg, 100 mg/kg, 120 mg/kg, 140 mg/kg, 150 mg/kg, 160 mg/kg, 180 mg/kg, 200 mg/kg, 225 mg/kg, 250 mg/kg, 275 mg/kg, 300 mg/kg, 325 mg/kg, 350 mg/kg, 375 mg/kg, 400 mg/kg, 450 mg/kg, 500 mg/kg, 550 mg/kg, 600 mg/kg, 700 mg/kg, 750 mg/kg, 800 mg/kg, 900 mg/kg, 1000 mg/kg, 1250 mg/kg, 1500 mg/kg, 1750 mg/kg, 2000 mg/kg, 2500 mg/kg, and/or 3000 mg/kg. Of course, all of these dosages are exemplary, and any dosage in-between these points is also expected to be of use in the invention, as are any ranges of dose defined by any two of these points. Any of the above dosage ranges or dosage levels may be employed for emodin alone or for emodin in combination with another anti-cancer drug or treatment.

“Therapeutically effective amounts” are those amounts effective to produce beneficial results in the recipient animal or patient. Such amounts may be initially determined by reviewing the published literature, by conducting in vitro tests or by conducting metabolic studies in healthy experimental animals. Before use in a clinical setting, it may be beneficial to conduct confirmatory studies in an animal model, preferably a widely accepted animal model of the particular disease to be treated. Preferred animal models for use in certain embodiments are rodent models, which are preferred because they are economical to use and, particularly, because the results gained are widely accepted as predictive of clinical value.

As is well known in the art, a specific dose level of active compounds such as emodin or emodin-like compounds for any particular patient depends upon a variety of factors including the activity of the specific compound employed, the age, body weight, general health, sex, diet, time of administration, route of administration, rate of excretion, drug combination, and the severity of the particular disease undergoing therapy. The person responsible for administration will, determine the appropriate dose for the individual subject. Moreover, for human administration, preparations should meet sterility, pyrogenicity, general safety and purity standards as required by FDA Office of Biologics standards.

A composition of the present invention is typically administered parenterally in dosage unit formulations containing standard, well known non-toxic physiologically acceptable carriers, adjuvants, and vehicles as desired. The term parental as used herein includes subcutaneous injections, intravenous, intramuscular, intra-arterial injection, or infusion techniques.

In some embodiments, the emodin or emodin-like compound will be administered in combination with a second agent. So long as a dose of second agent that does not exceed toxicity levels is not required, the effective amounts of the second agents may simply be defined as those amounts effective to reduce the cancer growth when administered to an animal in combination with the emodin-like agents. This is easily determined by monitoring the animal or patient and measuring those physical and biochemical parameters of health and disease that are indicative of the success of a given treatment. Such methods are routine in animal testing and clinical practice.

Examples of second agents that may be used with emodin or emodin-like tyrosine kinase inhibitor are anti-neoplastic agents. Examples of these are cisplatin; doxorubicin (Mechetner and Roninson, 1992) and analogues, such as 14-O-hemiesters of doxorubicin; etoposide; vincristine (Shirai et aL, 1994; Friche et al., 1993); vinblastine (Bear, 1994; McKinney and Hosford, 1993); actinomycin D (McKinney and Hosford, 1993); daunomycin (Bear, 1994); daunorubicin (Muller et al., 1994); taxotere (Hunter et al., 1993); taxol (Mechetner and Roninson, 1992); and tamoxifen (Trump et al., 1992). The skilled artisan is directed to “Physicians Desk Reference” 15th Edition, for dose ranges of chemotherapeutic agents practiced in the art. Some variation in dosage will necessarily occur depending on the condition of the subject being treated.

The treatment methods generally comprise administering to an animal with cancer, including a human patient, a therapeutically effective combination of emodin and/or emodin-like tyrosine kinase inhibitor alone in a lipid formulation or in combination with one or more second agents that is effective in treating cancer growth. The second agent(s) may be any of those listed above, and their functional equivalents.

### IV. Lipid:Emodin Formulations

In various embodiments of the invention, emodin and/or emodin derivatives may be associated with a lipid. Emodin and/or emodin derivatives associated with a lipid may be encapsulated in the aqueous interior of a liposome, interspersed within the lipid bilayer of a liposome, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. The lipid or lipid/emodin and/or emodin derivatives associated compositions of the present invention are not limited to any particular structure in solution. For example, they may be present in a bilayer structure, as micelles, or with a “collapsed” structure. They may also simply be interspersed in a solution, possibly forming aggregates which are not uniform in either size or shape.

Lipids are fatty substances which may be naturally occurring or synthetic lipids. For example, lipids include the fatty droplets that naturally occur in the cytoplasm as well as the class of compounds which are well known to those of skill in the art which contain long-chain aliphatic hydrocarbons and their derivatives, such as fatty acids, alcohols, amines, amino alcohols, and aldehydes. An example is the lipid dioleoylphosphatidylcholine (DOPC).

According to the present invention, phospholipids may be used for preparing lipid formulations (e.g., liposomes). Phospholipids may carry a net positive charge, a net negative charge or are neutral. Diacetyl phosphate can be employed to confer a negative charge on the inventive compositions, and stearylamine can be used to confer a positive charge on the compositions. The compositions, e.g., liposomes, can be made of one or more phospholipids.

In a particular embodiment, the lipid material is comprised of a neutrally charged lipid. A neutrally charged lipid can comprise a lipid without a charge, a substantially uncharged lipid or a lipid mixture with equal number of positive and negative charges.

In one aspect, the lipid component of the composition comprises a neutral lipid. In another aspect, the lipid material consists essentially of neutral lipids which is further defined as a lipid composition containing at least 70% of lipids without a charge. In other aspects, the lipid material may contain at least 80% to 90% of lipids without a charge. In yet other aspects, the lipid material may comprise about 90%, 95%, 96%, 97%, 98%, 99% or 100% lipids without a charge.

In certain aspects, the neutral lipid comprises a phosphatidylcholine, a phosphatidylglycerol, or a phosphatidylethanolamine. In a particular aspect, the phosphatidylcholine comprises DOPC.

In other aspects the lipid component comprises a substantially uncharged lipid. A substantially uncharged lipid is described herein as a lipid composition that is substantially free of anionic and cationic phospholipids and cholesterol. In yet other aspects the lipid component comprises a mixture of lipids to provide a substantially uncharged composition. Thus, the lipid mixture may comprise negatively and positively charged lipids.

Lipids suitable for use according to the present invention can be obtained from commercial sources. For example, dimyristyl phosphatidyl choline (“DMPC”) can be obtained from Sigma Chemical Co. (St. Louis, Mo.), dicetyl phosphate (“DCP”) is obtained from K and K Laboratories (Plainview, N.Y.); cholesterol (“Chol”) is obtained from Calbiochem-Behring; dimyristyl phosphatidyl glycerol (“DMPG”) and other lipids may be obtained from Avanti Polar Lipids, Inc. (Birmingham, Ala.). Stock solutions of lipids in chloroform or chloroform/methanol can be stored at about −20° C. Preferably, chloroform is used as the only solvent since it is more readily evaporated than methanol.

Phospholipids from natural sources, such as egg or soybean phosphatidylcholine, brain phosphatidic acid, brain or plant phosphatidylinositol, heart cardiolipin and plant or bacterial phosphatidylethanolamine are preferably not used as the primary phosphatide, i.e., constituting 50% or more of the total phosphatide composition, because of the instability and leakiness of the resulting liposomes.

“Liposome” is a generic term encompassing a variety of single and multilamellar lipid vehicles formed by the generation of enclosed lipid bilayers or aggregates. Liposomes may be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). However, the present invention also encompasses compositions that have different structures in solution than the normal vesicular structure. For example, the lipids may assume a micellar structure or merely exist as nonuniform aggregates of lipid molecules.

Liposomes used according to the present invention can be made by different methods. The size of the liposomes varies depending on the method of synthesis. A liposome suspended in an aqueous solution is generally in the shape of a spherical vesicle, having one or more concentric layers of lipid bilayer molecules. Each layer consists of a parallel array of molecules represented by the formula XY, wherein X is a hydrophilic moiety and Y is a hydrophobic moiety. In aqueous suspension, the concentric layers are arranged such that the hydrophilic moieties tend to remain in contact with an aqueous phase and the hydrophobic regions tend to self-associate. For example, when aqueous phases are present both within and without the liposome, the lipid molecules may form a bilayer, known as a lamella, of the arrangement XY-YX. Aggregates of lipids may form when the hydrophilic and hydrophobic parts of more than one lipid molecule become associated with each other. The size and shape of these aggregates will depend upon many different variables, such as the nature of the solvent and the presence of other compounds in the solution.

Liposomes within the scope of the present invention can be prepared in accordance with known laboratory techniques. A particular method of the invention describes the preparation of liposomes and is described below. Briefly, emodin or an emodin derivative is dissolved in t-butanol or DMSO and a phospholipid (Avanti Polar Lipids, Alabaster, Ala.), such as for example the phospholipid dimyristophosphatidylcholine (DMPC), is dissolved in tert-butanol. The lipid is then mixed with emodin or an emodin derivative. In the case of DMPC, the weight/weight ratio of the lipid to emodin or an emodin derivative is approximately 5:1 to 30:1. Tween 20 may be added to the lipid:emodin mixture such that tween 20 is 0.05 to 15% of the combined weight of the lipid, emodin and tween 20. Excess tert-butanol is added to this mixture such that the volume of tert-butanol is at least 95%. The mixture is vortexed, frozen in a dry ice/acetone bath and lyophilized overnight. The lyophilized preparation is stored at −20° C. and can be used up to three months. When required the lyophilized lipid formulation may be reconstituted in 0.9% saline. The average diameter of the particles obtained using TWeen 20 for encapsulating the lipid with the oligo is 0.7-1.0 μm in diameter.

Alternatively lipid formulations, such as liposomes, can be prepared by mixing lipids, in a solvent in a container, e.g., a glass, pear-shaped flask. The container should have a volume ten-times greater than the volume of the expected suspension of lipid formulation. Using a rotary evaporator, the solvent is removed at approximately 40° C. under negative pressure. The solvent normally is removed within about 5 min. to 2 hours, depending on the desired volume of the liposomes. The composition can be dried further in a desiccator under vacuum. The dried lipids generally are discarded after about 1 week because of a tendency to deteriorate with time.

Dried lipids can be hydrated at approximately 25-50 mM phospholipid in sterile, pyrogen-free water by shaking until all the lipid film is resuspended. The aqueous lipid formulation can be then separated into aliquots, each placed in a vial, lyophilized and sealed under vacuum.

In other alternative methods, lipid formulations can be prepared in accordance with other known laboratory procedures: the method of Bangham et al. (1965), the contents of which are incorporated herein by reference; the method of Gregoriadis, as described in DRUG CARRIERS IN BIOLOGY AND MEDICINE, G. Gregoriadis ed. (1979) pp. 287-341, the contents of which are incorporated herein by reference; the method of Deamer and Uster (1983), the contents of which are incorporated by reference; and the reverse-phase evaporation method as described by Szoka and Papahadjopoulos (1978). The aforementioned methods differ in their respective abilities to entrap aqueous material and their respective aqueous space-to-lipid ratios.

A pharmaceutical composition comprising the liposomes will usually include a sterile, pharmaceutically acceptable carrier or diluent, such as water or saline solution.

### V. Pharmaceuticals

Where clinical application of a lipid:emodin formulation is undertaken, it will be necessary to prepare the lipid:emodin or emodin derivative formulation as a pharmaceutical composition appropriate for the intended application. Generally, this will entail preparing a pharmaceutical composition that is essentially free of pyrogens, as well as any other impurities that could be harmful to humans or animals. One also will generally desire to employ appropriate buffers to render the complex stable and allow for uptake by target cells.

Aqueous compositions of the therapeutic composition of the present invention comprise an effective amount of emodin associated with a lipid as discussed above, further dispersed in pharmaceutically acceptable carrier or aqueous medium. The phrases “pharmaceutically” or “pharmacologically acceptable” refer to compositions that do not produce an adverse, allergic or other untoward reaction when administered to an animal, or a human, as appropriate.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients also can be incorporated into the compositions.

Solutions of therapeutic compositions can be prepared in water suitably mixed with a surfactant, such as hydroxypropylcellulose. Dispersions also can be prepared in glycerol, liquid polyethylene glycols, mixtures thereof and in oils. Under ordinary conditions of storage and use, these preparations contain a preservative to prevent the growth of microorganisms.

For human administration, preparations should meet sterility, pyrogenicity, general safety and purity standards as required by FDA Office of Biologics standards. The biological material may be extensively dialyzed to remove undesired small molecular weight molecules and/or lyophilized for more ready formulation into a desired vehicle, where appropriate. The active compounds will then generally be formulated for parenteral administration, e.g., formulated for injection via the intravenous, intramuscular, sub-cutaneous, intralesional, or even intraperitoneal routes. The preparation of an aqueous composition that contains the therapeutic composition as an active component or ingredient will be known to those of skill in the art in light of the present disclosure. Typically, such compositions can be prepared as injectables, either as liquid solutions or suspensions; solid forms suitable for using to prepare solutions or suspensions upon the addition of a liquid prior to injection can also be prepared; and the preparations can also be emulsified.

The pharmaceutical forms suitable for injection use include sterile aqueous solutions or dispersions; formulations including sesame oil, peanut oil or aqueous propylene glycol; and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi.

Pharmaceutically acceptable salts of compositions, include the acid addition salts and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier can also be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The therapeutic compositions of the present invention are advantageously administered in the form of injectable compositions either as liquid solutions or suspensions; solid forms suitable for solution in, or suspension in, liquid prior to injection may also be prepared. These preparations also may be emulsified. A typical composition for such purpose comprises a pharmaceutically acceptable carrier. For instance, the composition may contain 10 mg, 25 mg, 50 mg or up to about 100 mg of human serum albumin per milliliter of phosphate buffered saline. Other pharmaceutically acceptable carriers include aqueous solutions, non-toxic excipients, including salts, preservatives, buffers and the like.

Examples of non-aqueous solvents are propylene glycol, polyethylene glycol, vegetable oil and injectable organic esters such as ethyloleate. Aqueous carriers include water, alcoholic/aqueous solutions, saline solutions, parenteral vehicles such as sodium chloride, Ringer's dextrose, etc. Intravenous vehicles include fluid and nutrient replenishers. Preservatives include antimicrobial agents, anti-oxidants, chelating agents and inert gases. The pH and exact concentration of the various components the pharmaceutical composition are adjusted according to well known parameters.

Additional formulations are suitable for oral administration. Oral formulations include such typical excipients as, for example, pharmaceutical grades of mannitol, lactose, starch, magnesium stearate, sodium saccharine, cellulose, magnesium carbonate and the like. The compositions take the form of solutions, suspensions, tablets, pills, capsules, sustained release formulations or powders. When the route is topical, the form may be a cream, ointment, salve or spray.

The therapeutic compositions of the present invention may include classic pharmaceutical preparations. Administration of therapeutic compositions according to the present invention will be via any common route so long as the target tissue is available via that route. This includes oral, nasal, buccal, rectal, vaginal or topical. Alternatively, administration may be by orthotopic, intradermal subcutaneous, intramuscular, intraperitoneal or intravenous injection. Such compositions would normally be administered as pharmaceutically acceptable compositions that include physiologically acceptable carriers, buffers or other excipients. For treatment of conditions of the lungs, the preferred route is aerosol delivery to the lung. Volume of the aerosol is between about 0.01 ml and 0.5 ml. Similarly, a preferred method for treatment of colon-associated disease would be via enema. Volume of the enema is between about 1 ml and 100 ml.

An effective amount of the therapeutic composition is determined based on the intended goal. The term “unit dose” or “dosage” refers to physically discrete units suitable for use in a subject, each unit containing a predetermined-quantity of the therapeutic composition calculated to produce the desired responses, discussed above, in association with its administration, i.e., the appropriate route and treatment regimen. The quantity to be administered, both according to number of treatments and unit dose, depends on the protection desired.

Precise amounts of the therapeutic composition also depend on the judgment of the practitioner and are peculiar to each individual. Factors affecting the dose include the physical and clinical state of the patient, the route of administration, the intended goal of treatment (alleviation of symptoms versus cure) and the potency, stability and toxicity of the particular therapeutic substance.

Administration of the therapeutic construct of the present invention to a patient will follow general protocols for the administration of chemotherapeutics. It is expected that the treatment cycles would be repeated as necessary. It also is contemplated that various standard therapies, as well as surgical intervention, may be applied in combination with the described treatments.

According to the present invention, one may treat a cancer by directly injecting a tumor with the therapeutic composition of the present invention. Alternatively, the tumor or subject may be infused or perfused with a lipid:emodin formulation using any suitable delivery vehicle. Local or regional administration, with respect to the tumor, also is contemplated. Finally, systemic administration may be performed. Continuous administration also may be applied where appropriate, for example, where a tumor is excised and the tumor bed is treated to eliminate residual, microscopic disease. Delivery via syringe or catherization is preferred. Such continuous perfusion may take place for a period from about 1-2 hours, to about 2-6 hours, to about 6-12 hours, to about 12-24 hours, to about 1-2 days, to about 1-2 wk or longer following the initiation of treatment. Generally, the dose of the therapeutic composition via continuous perfusion will be equivalent to that given by a single or multiple injections, adjusted over a period of time during which the perfusion occurs.

In certain embodiments, the tumor being treated may not, at least initially, be resectable. Treatments with therapeutic compositions may increase the resectability of the tumor due to shrinkage at the margins or by elimination of certain particularly invasive portions. Following treatments, resection may be possible. Additional treatments subsequent to resection may serve to eliminate microscopic residual disease at the tumor site or in a subject.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. VI. Kits

All or portions of the essential materials and reagents required for inhibiting tumor or cancer cell proliferation may be assembled together in a kit. When the components of the kit are provided in one or more liquid solutions, the liquid solution preferably is an aqueous solution, with a sterile aqueous solution being particularly preferred.

For in vivo use, emodin or emodin-like compound, alone or in combination with, a chemotherapeutic agent may be formulated into a single or separate pharmaceutically acceptable syringeable composition. In this case, the container means may itself be an inhalant, syringe, pipette, eye dropper, or other such like apparatus, from which the formulation may be applied to an infected area of the body, such as the lungs, injected into an animal, or even applied to and mixed with the other components of the kit.

The components of the kit may also be provided in dried or lyophilized forms. When reagents or components are provided as a dried form, reconstitution generally is by the addition of a suitable solvent. It is envisioned that the solvent also may be provided in another container means. The kits of the invention may also include an instruction sheet defining administration of emodin.

The kits of the present invention also will typically include a means for containing the vials in close confinement for commercial sale such as, e.g., injection or blow-molded plastic containers into which the desired vials are retained. Irrespective of the number or type of containers, the kits of the invention also may comprise, or be packaged with, an instrument for assisting with the injection/administration or placement of the ultimate complex composition within the body of an animal. Such an instrument may be an inhalant, syringe, pipette, forceps, measured spoon, eye dropper or any such medically approved delivery vehicle.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

Material and Methods

This information is for a non-limiting exemplary lipid:emodin formulation or preparation. Emodin was prepared in t-butanol (Sigma St. Louis, Mo.) at a concentration of 1 mg/ml. T-butanol was warmed to 37° C., emodin was added and vortexed. The emodin/t-butanol mixture was then added to a lipid mixture. An exemplary lipid mixture is DMPC in t-butanol at concentration of 5 mg/ml. Other preparations were made using a combination of lipids (DMPC and DMPG) in ratios of 7:3, 5:2, and 1:1, but these preparations did not perform as well as DMPC alone. Tween 20 was added to the lipid mixture as a 10% solution (1 ml tween 20+9 ml t-butanol). In this particular example 300 μl of a 10% tween solution was added to the lipid mixture. The preparation therefore would have a 0.08% concentration of Tween 20 (0.3 ml×10% divided by 35.3 ml (total volume)=0.08%. Drying methods used were a lyophilizer and a speed vac. Both methods were utilized without heat and gave a similar product. The solvent used for reconstitution was saline (0.9%). PBS (phosphate buffered saline) was also used, but saline proved a more effective solvent.

The stability of the preparations was studied. Preparations stored at room temperature remained in solution over a period of time (8-48 hours) and if they became slightly cloudy this went back to clarity with vortexing and/or warming to approximately 37° C. Most preparations were stored in refrigerator or freezer if kept for extended periods of time. The product used in animal testing was made fresh before each series of injections. (DMPC=dimyristoyl phosphatidyl choline, DMPG=dimyristoyl phosphatidyl glycerol)

### Example 2

Emodin Treatment in C3H-HEJ Mouse Model

The leukemia strain 32D-bcr-abl was used, given at 1×10⁶ cells per CEH/HEJ mouse. The cell line is a 32D-P210 strain. The lipid:emodin formulation was run twice using an injection of the animal model of 0.2 mg emodin per animal. This formulation led to a product that was inconsistent with samples previously received by the Inventors from another laboratory. This prior formulation had been used for all of the cell culture work until this particular study. The product produced for this set of studies was likened to orange juice with pulp, as it had particulate matter that had not dissolved upon addition of saline. Injection into the mice was relatively easy, but reaction of the particulate in vivo was not definitive.

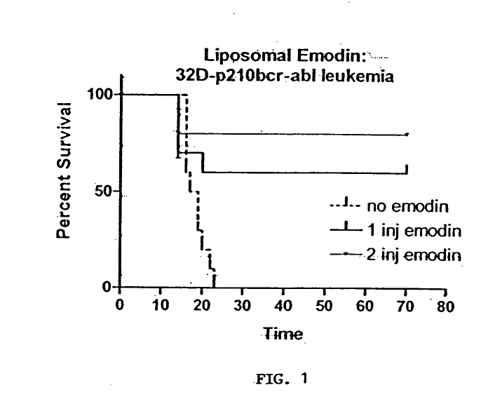

The results of the above described study was of 53 mice receiving on injection of 0.2 mg emodin, 8 died within the first two weeks of injection. Of the 51 that received two injections of 0.2 mg emodin, 9 died in the first two weeks. FIG. 1 illustrates an exemplary result of the study.

Leukemic 32D-bcr-abl cells were transplanted on day 0 into the congenic strain of mice CEH/HEJ. Animals receiving no treatment died between 18 and 22 days. Groups were treated with 1 or 2 injections of Liposomal emodin (8 mg/Kg of body weight) beginning on day 1. Liposomal emodin treated animals show a significant survival fraction—larger in the group receiving 2 injections of the agent than those receiving only 1 injection.

### Example 3

DMSO Solubilized Emodin Studies

In Vitro: A tetracycline regulatable 32D-P210 cell line was cultured in RPMI 1640 with 10% FCS and 10% Wehi supernatant and cultured at 37° C. Emodin was solubilized in DMSO at a concentration of 0.4 M for in vitro assays. A concentration of 3×10⁴ cells/ml were used in a 24 well plate for a 3 day exposure to emodin. Emodin was assayed at 60 μM, 30 μM, and 10 μM concentrations. At 72 hours, a MTT assay was performed to provide a colorometric analysis of cell growth.

In vivo: The mouse strain C3H-HEJ (8-10 weeks old) were used for in vivo studies. Emodin was prepared in an alkaline preparation at 10 mg/ml, and was administered to the animals via tail vein injections. One cohort of animals received a single dose of emodin equal to 43 mg/kg per animal. Two other cohorts received either two doses of drug, administered on consecutive days, or there doses of drug, administered on consecutive days. The total concentration of drug administered were 86 mg/kg and 129 mg/kg, respectively. Animals were followed until death, euthanized upon detection of illness, or euthanized at 100 days post leukemia injection.

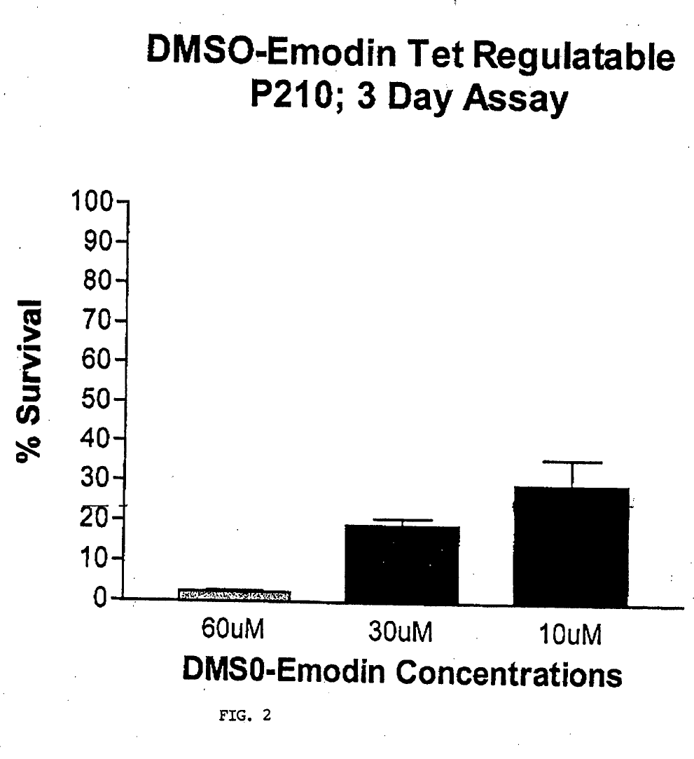

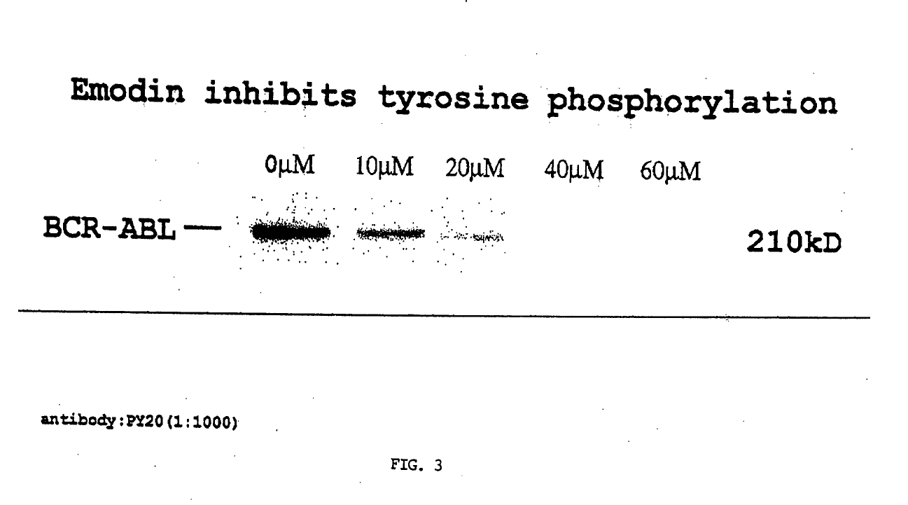

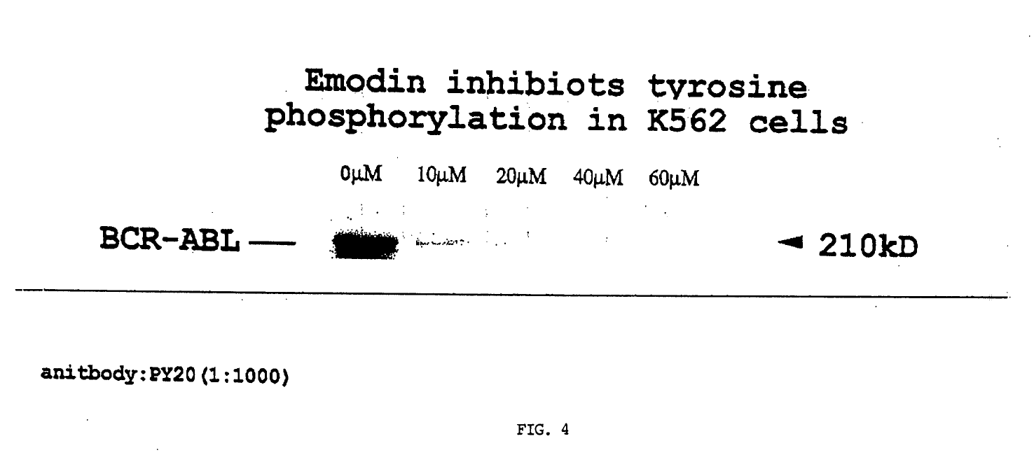

The in vitro assays have shown emodin to be an effective inhibitor of cellular growth, with an IC 50 of approximately 10 μM. See FIG. 2, 3, and 4.

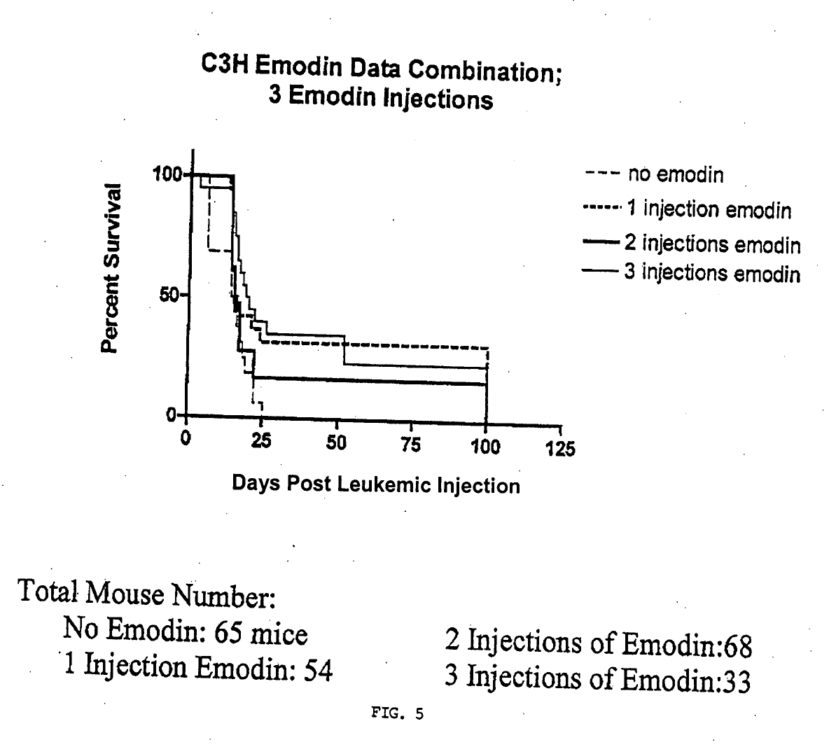

In vivo assays have shown that emodin can significantly increase the lifespan of animals injected with leukemia from 25 days for those animals that were untreated to 100 days when the animals were sacrificed. See FIG. 5. The alkaline preparation used in these studies presented the problem of a high pH solution to be injected into animals. Upon preparation, the solution had a pH of approximately 11, but could be lowered to approximately pH 10 for tail vein injection. Lowering the pH beyond the solubility of emodin with a precipitate forming in the solution. The preparation of pH 10 was moderately well tolerated by the animal, with some bruising of the tail being noted. Because of this a lipid:emodin formulation, as described above was developed.

### Example 4

Diffusion of Emodin Over Time

To determine the extent and rate at which emodin is released from the liposomal carrier, an in vitro experiment was carried out. The data in FIG. 6 demonstrate that within the initial 24 hr after addition of saline to the dried liposomal emodin preparation approximately 60% of the drug within liposomes was released. This increased to over 70% by 144 hr. These data demonstrate that the liposomal formulation of emodin provides for release of emodin rather than trapping or retaining the drug in the liposomes and secondly, the data demonstrate that the release rate is relatively slow. The later point is also important in that the LPE system represents a device that mimics a slow infusion of drug providing high sustained drug levels in comparison to a rapid rise and fall after, for example, simple intravenous administration of emodin itself.

An emodin:lipid composition was initially loaded with emodin at a concentration of 2.8 mg/mL. The diffusion of emodin over time was determined and plotted as concentration (mg/ml) in solution against time in hours. The diffusion data demonstrate that there is an approximate 70-75% release of material (emodin) over a prolonged period of time (FIG. 6).

### Example 5

Pharmacokinetic Studies of Emodin:Lipid Composition

The in vitro release rate data provide a good rationale for what is expected to happen upon full pharmacokinetic comparison of emodin versus liposomal emodin in mice. That is, that the LPE (or ELP) preparation can provide a slower release of drug than that from emodin injection providing a much greater area under the concentration-time curve (AUC) for drug from the LPE preparation (vs intravenous injection of free emodin).

This can be tested by pharmacokinetic studies in mice. Radiolabeled (³H-emodin) can be used to inject into mice directly or used to make the liposomal ³H-emodin preparation. These radiolabeled drug preparations can then be injected into mice and blood samples obtained over a 48 hr period of time. Plasma can be prepared from the whole blood. Under such a protocol, radioactivity in plasma from mice administered ³H-emodin will represent free ³H-emodin only. Radioactivity from mice injected with liposomal ³H-emodin can be derived from released ³H-emodin and ³H-emodin entrapped in residual liposomes. Both the free ³H-emodin and liposomal ³H-emodin can be separated using a radiometric detector connected to an HPLC system. The resulting data permits a comparison of the area under the curve (AUC) for free ³H-emodin from both preparations as well as the total release amount of ³H-emodin from the liposomal emodin preparation.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods in the steps or in the sequence of steps of the method described herein without departing from the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### References

The following references, to the extent that they provide exemplary procedural or other details supplementary to those set forth herein, are specifically incorporated herein by reference.
Bangham et al., J. Mol. Biol., 13:238, 1965.
Bear, Biochem. Biophys. Res. Commun., 200(1):513-521, 1994.
Friche et al., Br. J. Cancer, 67(2):226-231, 1993.
Ghosh and Bachhawat, In: Liver Diseases, Targeted Diagnosis and Therapy Using Specific Receptors and Ligands. Wu et aL (eds), Marcel Dekker, NY, 87-104, 1991.
Gregoriadis, Drug Carriers in Biology and Medicine, Gregoriadis (ed.), 1979.
Hunter et al., Pharm. Res., 10(5):743-749, 1993.
Jayasuriya et al., J. Nat. Prod., 55:696-698, 1992.
Juliano, et al., In: Drug Delivery Systems, Juliano (ed)., Oxford Press, NY, 189-230, 1980.
Kupchan and Karim, Lloydia, 39:223-224, 1976.
McKinney and Hosford, J. Biol. Chem., 268(10):6886-6895, 1993.
Mechetner and Roninson, Proc. Natl. Acad. Sci. USA, 89(13):5824-5828, 1992.
Muller et al., FEBS Lett, 343(2):68-72, 1994.
Remington's Pharmaceutical Sciences, 15th Edition, 61:1035-1038 and 1570-1580, 1980.
Shirai et al., Biochim Biophys Acta, 1222(3):400-404, 1994.
Szoka and Papahadjopoulos, Proc. Natl. Acad. Sci., 75:4194-4198, 1978.
Trump et al., J. Natl. Cancer Inst., 84(23):1811-1816, 1992.
Yeh et al., Planta Med., 54:413-414, 1988.

1. A composition comprising emodin, or a derivative thereof, associated with a lipid.

2. The composition of claim 1, wherein the lipid comprises dimyristol phosphatidyl choline.

3. The composition of claim 1, wherein the lipid comprises dimyristol phosphatidyl glycerol.

4. The composition of claim 1, wherein a weight/weight ratio of lipid to emodin is about 5:1 to about 30:1.

5. The composition of claim 4, wherein the ratio of lipid to emodin is about 5:2.

6. The composition of claim 1, further comprising a solubilizing agent.

7. The composition of claim 6, wherein the solubilizing agent is a non-ionic detergent.

8. The composition of claim 7, comprising an amount of non-ionic detergent of about 0.05 to 15 percent weight/weight.

9. The composition of claim 8, wherein the amount of non-ionic detergent is about 0.08 percent weight/weight.

10. The composition of claim 7, wherein the non-ionic detergent is tween.

11. The composition of claim 10, wherein tween is tween 20.

12. The composition of claim 6, wherein the solubilizing agent is soybean or peanut oils.

13. The composition of claim 6, wherein the solubilizing agent is a β-hydroxylated compound.

14. A method of preparing a composition, comprising the step of admixing emodin or a derivative thereof with a lipid in a solvent.

15. The method of claim 14, wherein the lipid is dimyristol phosphatidyl choline.

16. The method of claim 14, wherein a weight/weight ratio of lipid to emodin is about 5:1 to about 30:1.

17. The method of claim 16, wherein the ratio of lipid to emodin is about 15:1.

18. The method of claim 14, wherein the solvent is tertiary butanol.

19. The method of claim 14, further comprising admixing a solubilization agent.

20. The method of claim 19, wherein the solubilization agent is approximately 0.05 to 15% of the composition.

21. The method of claim 20, wherein the solubilization agent is approximately 0.08% of the composition.

22. The method of claim 19, wherein the solubilization agent is tween 20.

23. The method of claim 19, wherein the solubilization agent is soy or peanut oils.

24. The method of claim 19, wherein the solubilization agent is a β-hydroxylated compound.

25. The method of claim 14, further comprising lyophilizing the composition.

26. The method of claim 25, further comprising reconstituting the lyophilized composition in a solvent.

27. The method of claim 26, wherein the solvent is a saline solution.

28. The method of claim 27, wherein the saline solution is a 0.9% saline solution.

29. A method for treating cancer in a subject comprising administering to said subject a formulation comprising emodin associated with a lipid (lipid:emodin formulation), wherein a tyrosine kinase activity in cancer cells is inhibited in said subject.

30. The method of claim 29, wherein emodin is provided in a dose of approximately 1 mg/Kg of body weight to approximately 50 mg/Kg of body weight.

31. The method of claim 29, wherein the cancer is a hematopoetic cancer.

32. The method of claim 31, wherein the hematopoetic cancer is leukemia.

33. The method of claim 29, wherein administration of the lipid:emodin formulation is by injection.

34. The method of claim 33, wherein administration of the lipid:emodin formulation is by intravascular injection.

35. The method of claim 29, wherein the lipid:emodin formulation is administered at least once.

36. The method of claim 29, wherein the lipid:emodin formulation is administered at least twice.

37. The method of claim 29, wherein the lipid:emodin formulation is administered at least three times.
